(19) United States
(12) Patent Application Publication
(10) Pub. No.: US 2025/0329462 A1
(43) Pub. Date: Oct. 23, 2025

(54) METHOD, DEVICE AND PROGRAM FOR PROVIDING SERVICE TO DETERMINE ACTIVITY LEVEL OF DISEASE

(71) Applicant: Min Seok CHANG, Wonju-si (KR)

(72) Inventor: Min Seok CHANG, Wonju-si (KR)

(21) Appl. No.: 19/254,682

(22) Filed: Jun. 30, 2025

Related U.S. Application Data
(63) Continuation of application No. PCT/KR2023/014159, filed on Sep. 19, 2023.

(30) Foreign Application Priority Data
Dec. 29, 2022 (KR) 10-2022-0188758

(51) Int. Cl. G16H 50/20; G16H 10/20; G16H 70/60; G16H 80/00

(52) U.S. Cl. CPC G16H 50/20; G16H 10/20; G16H 70/60; G16H 80/00

(57) ABSTRACT

The present disclosure relates to a method, device, and program for providing a service to determine the activity level of a disease on the basis of responses to question information designed to evaluate the activity level of a disease that a user possesses, thereby allowing for quick intervention by a doctor according to the determined activity level. The method includes: collecting symptom information for various diseases; generating multiple pieces of question information and a list of answers for evaluating the activity level for each of the various diseases on the basis of the collected symptom information; acquiring information about the disease that a user possesses; providing the user with question information and an answer list corresponding to the disease that the user possesses; and determining the activity level of the disease that the user possesses on the basis of the answers to the question information obtained from the user.

### CROSS REFERENCE TO RELATED APPLICATIONS

This application is a Continuation of International Application No. PCT/KR2023/014159, filed on Sep. 19, 2023, which claims the benefit of Korean Patent Application No. 10-2022-0188758, filed on Dec. 29, 2022, in the Korean Intellectual Property Office, the disclosure of which is incorporated herein in its entirety by reference.

### TECHNICAL FIELD

The present invention relates to a service provision method, device, and program for determining activity of a disease, and more particularly, to a service provision method, device, and program for determining activity of a disease that evaluate activity of a user's disease on the basis of answers to question information for evaluating activity of the disease and enable a doctor to rapidly intervene in accordance with the activity.

### BACKGROUND ART

These days, medical services are becoming more specialized, fractionized, and complex, but acquisition of information and choice of a medical facility and a healthcare provider for healthcare, self-management, diagnosis, treatment, and the like have not kept pace with services.

In other words, patients generally infer related diseases or current health conditions from their perceived symptoms and take appropriate self-care measures or choose a medical facility and a healthcare provider on their own or on the advice of others. Also, patients should determine activity of their diseases, but it is difficult for patients to determine activity on their own.

Therefore, even when a symptom of a patient's disease changes, it is difficult to know whether this is a natural healing process or a worsening process of the disease, and an appropriate time for treatment may be missed accordingly. Therefore, it is a pressing issue for patients to determine an individual's disease status in real time and take necessary measures.

### DISCLOSURE

### Technical Problem

An object to be solved by the present invention is to provide a service provision method, device, and program for determining activity of a disease in which activity of a user's disease is evaluated on the basis of answers to question information for evaluating activity of the disease and a doctor can rapidly intervene in accordance with the activity.

Objects to be achieved by the present invention are not limited to that described above, and other objects that have not been described will be clearly understood by those skilled in the art from the following description.

### Technical Solution

To achieve the above-described purpose, a service provision method for determining activity of a disease according to an embodiment of the present invention includes collecting symptom information about various diseases, generating a plurality of pieces of question information and a plurality of answer lists for evaluating activity of each of the various diseases on the basis of the collected symptom information, acquiring information on a user's disease, providing the user with question information and answer lists corresponding to the user's disease, and determining activity of the user's disease on the basis of answers to the question information acquired from the user.

The generating of the plurality of pieces of question information and the plurality of answer lists may include extracting symptom information common to several diseases from symptom information about the various diseases, generating question information about symptom information not common to several diseases and the common symptom information in the symptom information, generating a plurality of answer lists corresponding to the question information, and classifying the generated question information and the answer lists by disease.

The determining of the activity of the user's disease may include receiving at least one selected from a plurality of answers to each piece of the question information from the user, when answers to all the question information are input, calculating a score corresponding to an answer selected for each piece of the question information and integrating the calculated scores, and comparing the integrated score with a score integrated on the basis of answers to the question information previously acquired from the user and determining activity of the user's disease in accordance with a comparison result.

The integrating of the calculated scores may include extracting weights set for the question information, calculating scores by applying the weights set for the question information to scores corresponding to the selected answers to the question information, and integrating the scores to which the weights are applied.

The determining of the activity of the user's disease may include predicting a change in the activity of the user's disease on the basis of an answer to a key question for determining the activity of the disease, and when the activity of the user's disease is predicted to increase, finally determining a change in the activity of the user's disease on the basis of answers to auxiliary questions. The final determining of the change in the activity of the disease may further include determining that the user's disease has worsened when the activity of the disease is determined to have increased as a result of analyzing the answers to the auxiliary questions, and determining that the user's disease has improved when the activity of the disease is determined to have decreased.

The generating of the plurality of pieces of question information and the plurality of answer lists may include separating the common symptom information into a plurality of sections in accordance with symptom severity and matching a disease to symptom severity corresponding to each section to store the corresponding data. The determining of the activity of the user's disease may include extracting an answer to the question information corresponding to the common symptom information from the answers to the question information acquired from the user, when the extracted answer corresponds to symptom severity of a disease other than the user's disease, providing question information and an answer list corresponding to the other disease to the user, and determining whether the user has the other disease on the basis of an answer to the question information acquired from the user.

The acquiring of the information on the user's disease may further include acquiring a measurement value using a measurement device owned by the user in accordance with a type of the user's disease, and the determining of the activity of the user's disease may further include comparing the measurement value with a measurement value previously acquired from the user using the measurement device to determine activity of the user's disease.

The service provision method may further include providing the determined activity of the disease to the user and providing the determined activity of the disease to a hospital or a doctor designated by the user in accordance with the activity of the disease.

To achieve the above-described purpose, a service provision device for determining activity of a disease according to an embodiment of the present invention includes a memory configured to store one or more instructions and a processor configured to execute the one or more instructions stored in the memory. The processor may execute the one or more instructions to perform a method of providing a service to determine activity of a disease.

To achieve the above-described purpose, a service provision program for determining activity of a disease according to an embodiment of the present invention is stored in a computer-readable recording medium to perform a method of providing a service to determine activity of a disease in combination with a computer which is hardware.

Other details of the present invention are included in the detailed description and drawings.

### Advantageous Effects

According to the present invention, it is possible to determine whether each of several diseases has developed on the basis of answers to question information for determining whether various diseases have developed.

In addition, when it is determined that a user has a disease, it is possible to transmit the user's condition to a hospital or a doctor such that a treatment service may be provided.

Effects of the present invention are not limited to those described above, and other effects that have not been described will be clearly understood by those skilled in the art from the following description.

### DESCRIPTION OF DRAWINGS

FIG. 1 is a diagram showing a service provision system for determining activity of a disease according to an embodiment of the present invention.

FIG. 2 is a hardware configuration diagram of a service provision device according to an embodiment of the present invention.

FIG. 3 is a diagram illustrating a service provision method for determining activity of a disease according to an embodiment of the present invention.

FIG. 4 is a diagram illustrating a method of generating a plurality of pieces of question information and a plurality of answer lists on the basis of collected symptom information according to an embodiment of the present invention.

FIG. 5 is a diagram illustrating a method of determining activity of a disease according to a first embodiment of the present invention.

FIG. 6 is a diagram illustrating a method of determining activity of a disease according to a second embodiment of the present invention.

FIG. 7 is a diagram illustrating a method of determining whether a user has a disease according to another embodiment of the present invention.

### MODES OF THE INVENTION

The advantages and features of the present invention and a method of achieving them will become apparent from embodiments which will be described in detail below with reference to the accompanying drawings. However, the present invention is not limited to the embodiments disclosed below and may be implemented in various different forms. The embodiments are only provided to make the disclosure of the present invention complete and fully convey the scope of the present invention to those skilled in the technical field to which the present invention pertains. The present invention is only defined by the scope of the claims.

Terminology used herein is only for the purpose of describing embodiments and is not intended to limit the present invention. In this specification, singular forms include plural forms as well unless the context particularly indicates otherwise. The terms “comprises” and/or “comprising” used in this specification do not preclude the presence or addition of one or more components other than stated components. Throughout the specification, like reference numerals refer to like components, and the term “and/or” includes any of stated components or a combination of one or more stated components. Although the terms “first,” “second,” and the like may be used to describe various components, these components are not limited by the terms. These terms are used for the sole purpose of distinguishing one component from others. Therefore, a first component mentioned below may be a second component within the technical scope of the present invention.

Unless defined otherwise, all terms used herein (including technical or scientific terms) have the same meanings as those generally understood by those skilled in the technical field to which the present invention pertains. In addition, terms defined in commonly used dictionaries are not interpreted ideally or excessively unless clearly and particularly defined herein.

As used herein, the term “unit” or “module” refers to a software component or a hardware component such as a field programmable gate array (FPGA) or an application-specific integrated circuit (ASIC), and a “unit” or “module” performs certain roles. However, a “unit” or “module” is not limited to software or hardware. A “unit” or “module” may be configured to reside in an addressable storage medium or run on one or more processors. Accordingly, as an example, a “unit” or “module” includes components, such as software components, object-oriented software components, class components, and task components, processes, functions, attributes, procedures, subroutines, segments of program code, drivers, firmware, microcode, circuitry, data, databases, data structures, tables, arrays, and variables. Components and functions provided in “units” or “modules” may be combined into a smaller number of components, and “units” or “modules” may be subdivided into additional components and “units” or “modules.”

In this specification, a computer is any type of hardware device including at least one processor and may be understood as collectively including a software configuration operating in the corresponding hardware device according to embodiments. For example, a computer may be understood as, but is not limited to, a meaning including all of a smartphone, a tablet personal computer (PC), a desktop computer, a laptop computer, and a user client and an application running on each device.

Hereinafter, embodiments of the present invention will be described in detail with reference to the accompanying drawings.

Each step described in this specification is described as being performed by a computer, but the subject of each step is not limited thereto. At least a part of each step may be performed in different devices according to embodiments.

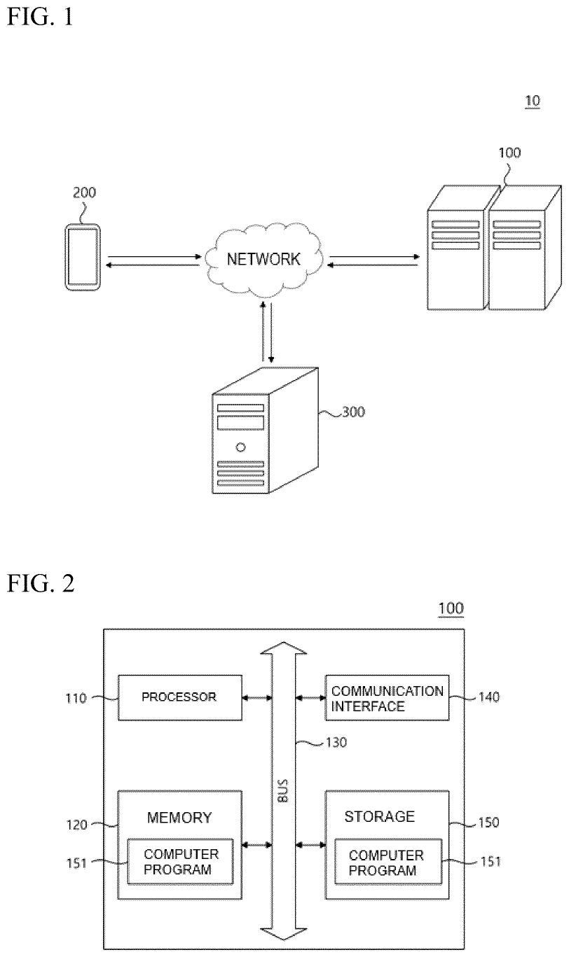

FIG. 1 is a diagram showing a service provision system for determining activity of a disease according to an embodiment of the present invention.

Referring to FIG. 1, the service provision system for determining activity of a disease according to an embodiment of the present invention may include a service provision device 100, a user terminal 200, and an external server 300.

Here, the service provision system for determining activity of a disease shown in FIG. 1 is in accordance with an embodiment, and components thereof are not limited to the embodiment shown in FIG. 1 and may be added, changed, or removed as necessary.

The service provision device 100 may collect symptom information about various diseases, generate a plurality of pieces of question information and a plurality of answer lists for evaluating activity of each of the various diseases on the basis of the collected symptom information, acquire information on a user's disease, provide question information and an answer list corresponding to the user's disease to the user, and determine activity of the user's disease on the basis of answers acquired from the user.

The user terminal 200 may be connected to a website via a network and receive a service for determining activity of a disease from the service provision device 100.

The user terminal 200 may be at least one of a smartphone, a tablet PC, a desktop computer, and a laptop computer having a display in at least a part thereof and may receive a service for determining activity of a disease provided by the service provision device 100 during the process of executing a browser. However, the user terminal 200 is not limited thereto.

The user terminal 200 may be connected to the service provision device 100 and receive various services and platforms from the service provision device 100.

The user terminal 200 may receive various services provided by the service provision device 100 by downloading, installing, and executing an application for the various services and platforms provided by the service provision device 100. To this end, an operating system (OS) for running the application may be installed on the user terminal 200 like a smartphone. However, the user terminal 200 is not limited thereto and may be another general-use device for running the application.

In various embodiments, the user terminal 200 may provide a service on the basis of the web rather than the application, and a method with which the user terminal 200 provides a service is not limited to any particular format.

The external server 300 may be connected to the service provision device 100 via the network and may store and manage various information for the service provision device 100 to perform a service provision method for determining activity of a disease.

Also, the external server 300 may receive and store various information and data that is generated when the service provision device 100 performs the service provision method for determining activity of a disease. For example, the external server 300 may be a storage server separately provided outside the service provision device 100. A hardware configuration of the service provision device 100 will be described with reference to FIG. 2.

FIG. 2 is a hardware configuration diagram of a service provision device according to an embodiment of the present invention.

Referring to FIG. 2, the service provision device (hereinafter, “computing device”) 100 may include at least one processor 110, a memory 120 into which a computer program 151 executed by the processor 110 is loaded, a bus 130, a communication interface 140, and a storage 150 in which the computer program 151 is stored. In FIG. 2, only components related to embodiments of the present invention are shown. Therefore, those skilled in the technical field to which the present invention pertains should appreciate that general-use components other than the components shown in FIG. 2 may be additionally included.

The processor 110 controls overall operations of each component of the computing device 100. The processor 110 may include a central processing unit (CPU), a micro-processor unit (MPU), a micro-controller unit (MCU), a graphics processing unit (GPU), or any form of processor well known in the technical field of the present invention.

In addition, the processor 110 may perform computation for at least one application or program for executing methods according to embodiments of the present invention, and the computing device 100 may include at least one processor.

In various embodiments, the processor 110 may further include a random access memory (RAM; not shown) and a read-only memory (ROM; not shown) which temporarily and/or permanently store a signal (or data) processed in the processor 110. Also, the processor 110 may be implemented in the form of a system on chip (SoC) including at least one of a GPU, a RAM, and a ROM.

The memory 120 stores various data, commands, and/or information. The computer program 151 may be loaded from the storage 150 into the memory 120 to perform methods/operations according to various embodiments of the present invention. When the computer program 151 is loaded into the memory 120, the processor 110 may perform the methods/operations by executing one or more instructions constituting the computer program 151. The memory 120 may be implemented as a volatile memory, such as a RAM, but the technical scope of the present disclosure is not limited thereto.

The bus 130 provides a communication function between the components of the computing device 100. The bus 130 may be implemented in one of various forms of buses such as an address bus, a data bus, a control bus, and the like.

The communication interface 140 supports wired or wireless Internet communication of the computing device 100. Also, the communication interface 140 may support various communication methods in addition to Internet communication. To this end, the communication interface 140 may include a communication module well known in the technical field of the present invention. In some embodiments, the communication interface 140 may be omitted.

The storage 150 may non-temporarily store the computer program 151. When the computing device 100 performs a service provision method for determining activity of a disease, the storage 150 may store various information required for providing the service provision method for determining activity of a disease.

The storage 150 may include a non-volatile memory, such as a ROM, an erasable programmable ROM (EPROM), an electrically erasable programmable ROM (EEPROM), a flash memory, and the like, a hard disk, a removable disk, or any form of computer-readable recording medium well known in the technical field to which the present invention pertains.

The computer program 151 may include one or more instructions that cause the processor 110 to perform methods/operations according to various embodiments of the present invention when loaded into the memory 120. In other words, the processor 110 may perform the methods/operations according to various embodiments of the present invention by executing the one or more instructions.

In an embodiment, the computer program 151 may include one or more instructions for performing a service provision method for determining activity of a disease including a step of collecting symptom information about various diseases, a step of generating a plurality of pieces of question information and a plurality of answer lists for evaluating activity of each of the various diseases on the basis of the collected symptom information, a step of acquiring information on a user's disease, a step of providing the user with question information and an answer list corresponding to the user's disease, and a step of determining activity of the user's disease on the basis of answers to the question information acquired from the user.

Steps of the methods or algorithms described in connection with embodiments of the present invention may be directly implemented using hardware, implemented using software modules executed by hardware, and implemented using a combination thereof. The software modules may reside in a RAM, a ROM, an EPROM, an EEPROM, a flash memory, a hard disk, a removable disk, a compact disc (CD)-ROM, or any form of a computer-readable recording medium well known in the technical field to which the present invention pertains.

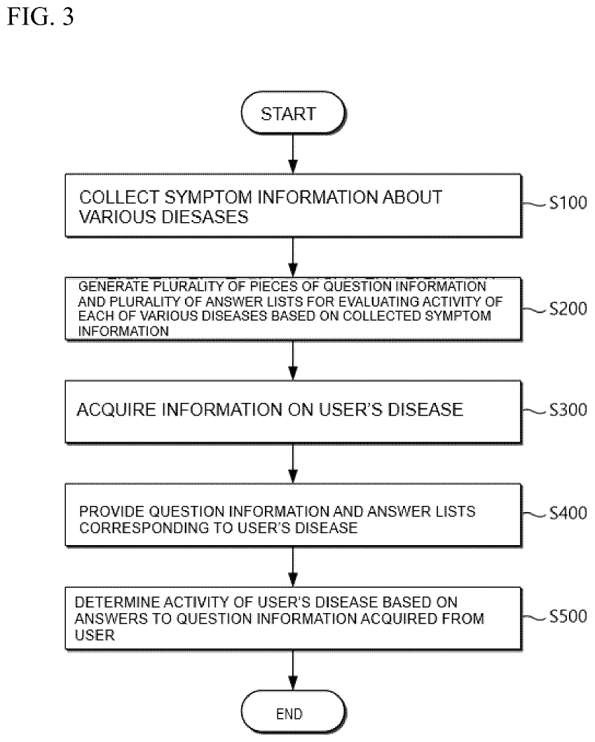

Components of the present invention may be implemented as a program (or application) and stored in a medium to be executed in combination with a computer which is hardware. Components of the present invention may be executed by software programming or software elements. Similarly, embodiments may be implemented in a programming or scripting language, such as C, C++, Java, an assembler, or the like, to include various algorithms which are embodied as combinations of data structures, processes, routines, or other programming elements. Functional aspects may be implemented as an algorithm executed by one or more processors. A service provision method for determining activity of a disease provided by the computing device 100 will be described below with reference to FIG. 3.

FIG. 3 is a diagram illustrating a service provision method for determining activity of a disease according to an embodiment of the present invention.

Referring to FIG. 3, the computing device 100 may collect symptom information about various diseases (S100). The computing device 100 may collect symptom information about various diseases through web crawling and collect symptom information in accordance with a manager's input. A method for the computing device 100 to collect symptom information about various diseases is not limited thereto and may be performed in accordance with various embodiments.

The computing device 100 may collect symptom information about asthma, which may include wheezing, shortness of breath, and coughing. Also, the computing device 100 may collect symptom information about each of obstructive pulmonary disease, heart failure, and renal failure. The symptom information about obstructive pulmonary disease may include shortness of breath, coughing, sputum, wheezing, and chest tightness, the symptom information about heart failure may include shortness of breath, chronic fatigue, and indigestion, and the symptom information about renal failure may include enuresis, sleep disturbance, indigestion, vomiting, fatigue, decreased urine output, edema, dyspnea, headache, weight loss, convulsions, and coma. However, diseases about which the computing device 100 collects information are not limited thereto, and symptom information about diseases may not be limited thereto either.

The computing device 100 may generate a plurality of pieces of question information and a plurality of answer lists for evaluating activity of each of various diseases on the basis of the collected symptom information (S200).

The computing device 100 may generate a plurality of pieces of question information for evaluating activity on the basis of the collected symptom information about diseases and generate a list of a plurality of answers to each piece of question information.

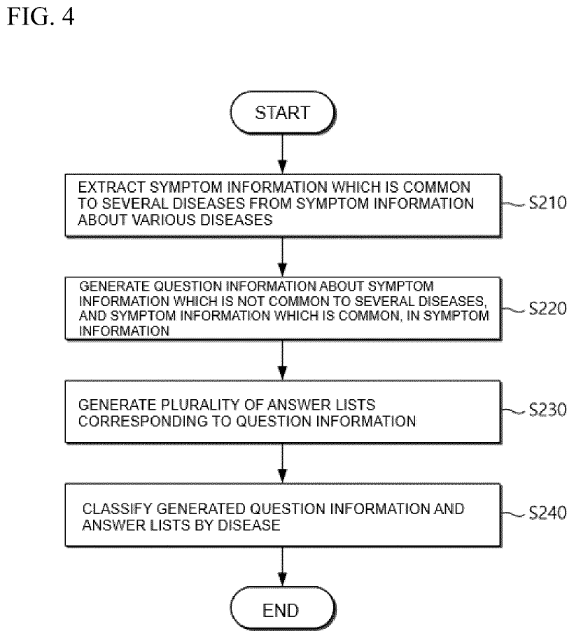

A detailed method of generating a plurality of pieces of question information and a plurality of answer lists for evaluating activity on the basis of collected symptom information will be described with reference to FIG. 4.

FIG. 4 is a diagram illustrating a method of generating a plurality of pieces of question information and a plurality of answer lists on the basis of collected symptom information according to an embodiment of the present invention.

Referring to FIG. 4, the computing device 100 may extract symptom information which is common to several diseases from symptom information about various diseases (S210). For example, symptom information about obstructive pulmonary disease includes shortness of breath, coughing, sputum, wheezing, and chest tightness and symptom information about heart failure includes shortness of breath, ankle swelling, chronic fatigue, and indigestion. Here, obstructive pulmonary disease and heart failure may include the same symptom information of shortness of breath. The computing device 100 may extract shortness of breath as common symptom information. The computing device 100 may extract common symptom information from at least two diseases.

The computing device 100 may generate question information about symptom information which is not common to several diseases and symptom information which is common in the symptom information (S220). The computing device 100 may generate question information about each piece of the symptom information and determine common symptom information as one piece of symptom information to generate question information.

For example, when shortness of breath is extracted as the same symptom information about obstructive pulmonary disease and heart failure as described above, the computing device 100 may generate question information about each of shortness of breath, coughing, sputum, wheezing, chest tightness, ankle swelling, chronic fatigue, and indigestion.

Meanwhile, when common question information is not extracted, the computing device 100 may generate question information about shortness of breath which is a symptom of obstructive pulmonary disease and question information about shortness of breath which is a symptom of heart failure. In this case, identical or similar questions are generated, and thus a user may have to answer the same question several times, which is inconvenient.

To solve this problem, the computing device 100 may generate the same question information about common symptom information rather than generating question information about the same symptom information for each disease. Accordingly, it is possible to not only reduce the load required for generating questions but also reduce the hassle of having to select an answer to the same question several times from the user's perspective.

The computing device 100 may generate a plurality of answer lists corresponding to the question information (S230). For example, the question information may be intended to determine whether the user has a disease on the basis of symptom information, and may include a question such as “How often have you been coughing?,” “How often have you been out of breath in the past week?,” or the like. Accordingly, the computing device 100 may generate a list of a plurality of answers for answering the question information. For example, as a list of answers to “How often have you been coughing?,” a list of a plurality of answers such as at least once a day, 3 to 6 times a week, 1 to 2 times a week, and never may be generated. Here, the frequencies and the period in the answer list may be determined in accordance with an indicator for determining severity of a corresponding disease but are not limited by the indicator. In addition, the number of answers in the list may not be limited thereto, and lists with different numbers of answers may be generated in accordance with question information.

The computing device 100 may classify the generated question information and answer lists by disease (S240). For example, generated question information about shortness of breath and a plurality of answer lists may be classified as question information and answer lists for each of obstructive pulmonary disease and heart failure. In other words, question information about obstructive pulmonary disease may include the generated question information about shortness of breath, and question information about heart failure may also include the generated question information about shortness of breath.

Referring back to FIG. 3, the computing device 100 may acquire information on the user's disease (S300). The computing device 100 may provide an instruction to select or input the user's disease, and the user may select or input information on his or her disease in accordance with the instruction provided via the user terminal 200.

The computing device 100 may provide question information and answer lists corresponding to the user's disease to the user (S400) and determine activity of the user's disease on the basis of answers to the question information acquired from the user (S500).

The user may check the question information and answer lists provided via the user terminal 200 and select answers to questions in accordance with an instruction provided by the computing device 100. The computing device 100 may provide the instruction to select at least one answer to every piece of the question information.

The computing device 100 may analyze a list of answers selected by the user to determine activity of the user's disease. For example, when the user is suffering from heart failure, the computing device 100 may provide question information and answer lists for heart failure to the user and analyze answers acquired from the user to determine whether symptoms of heart failure have improved or worsened.

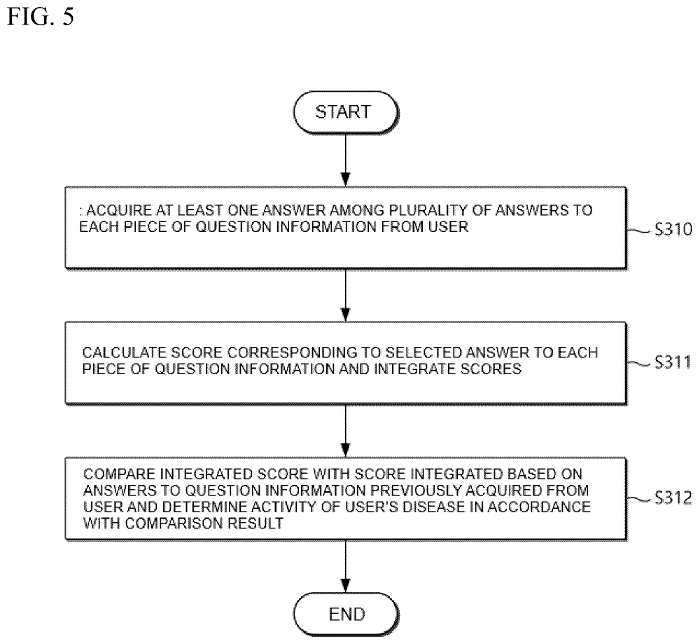

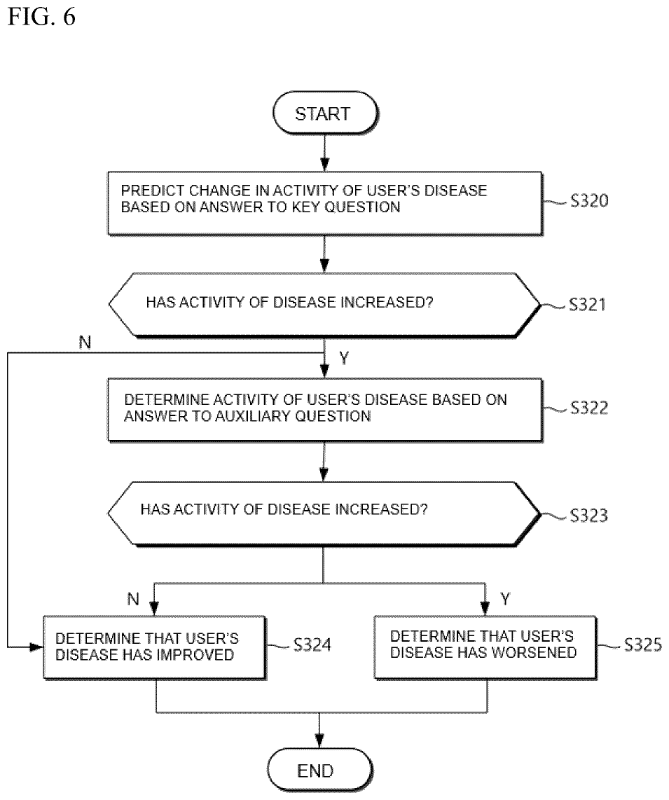

A detailed method of determining activity of a user's disease will be described with reference to FIGS. 5 and 6.

FIG. 5 is a diagram illustrating a method of determining activity of a disease according to a first embodiment of the present invention.

Referring to FIG. 5, the computing device 100 may acquire at least one of a plurality of answers to each piece of question information from the user (S310).

When answers to all questions are input, the computing device 100 may calculate a score corresponding to a selected answer for each piece of question information and integrate the calculated scores (S311). The computing device 100 may set and store a score for each answer and read a score corresponding to an answer selected by the user.

The computing device 100 may integrate all scores of answers to the question information. Here, integrating scores may be summing or averaging all the scores or may be integrating scores calculated in accordance with weights.

The computing device 100 may store a weight corresponding to each piece of question information and calculate a score in accordance with the weight. For example, when a wheezing sound is heard, it is likely due to the onset of asthma, while coughing or shortness of breath is less likely to be due to the development of asthma. In other words, since coughing or shortness of breath is a symptom that may result from reasons other than asthma, it may be difficult to determine that the user has asthma just because he or she coughs or has shortness of breath. Accordingly, the computing device 100 may assign a high weight to question information about wheezing and assign a low weight to question information about coughing or shortness of breath. Meanwhile, in connection with asthma, it has been described that a higher weight is assigned to question information about wheezing, but the present invention is not limited thereto.

In other words, the computing device 100 may apply weights corresponding to question information to scores corresponding to answers selected by the user and integrate the scores to which the weights are applied. Here, applying weights may be, but is not limited to, multiplying scores by the weights and summing or averaging all the scores to which the weights are applied.

In addition, different scores may be set for answer lists depending on a weight of each question. For example, a list of answers to question information about wheezing may have scores of 5, 10, 15, and 20, and a list of answers to question information about coughing may have scores of 1, 2, 3, and 4. This may reflect that a weight applied to question information about wheezing is higher than a weight applied to question information about coughing.

The computing device 100 may compare the integrated score with a score integrated on the basis of the user's previous answers to the question information and determine activity of the user's disease in accordance with the comparison result (S312). The computing device 100 may periodically provide a service for determining activity of a disease to the user. For example, the computing device 100 may provide question information and answer lists to the user at certain intervals, calculate and integrate a score every time an answer to the question information is acquired from the user, and store answer results and scores. Accordingly, the computing device 100 may compare a newly acquired answer to question information with a previous answer. Here, the previous answer may be, but is not limited to, an answer that is acquired just before the newly acquired answer to the question information.

The computing device 100 may compare a score calculated for each answer to each piece of question information with a previous score or compare the integrated score with a previous integrated score to determine activity of the disease. For example, when the currently acquired integrated score for the question information is higher than a previously acquired integrated score for the question information, it may be determined that the disease has worsened, and when the currently acquired integrated score is lower than the previously acquired integrated score, it may be determined that the disease has improved. However, the present invention is not limited thereto. When the currently acquired integrated score for the question information is lower than a previously acquired integrated score for the question information, it may be determined that the disease has worsened, and when the currently acquired integrated score is higher than the previously acquired integrated score for the question information, it may be determined that the disease has improved. In addition, the computing device 100 may set a certain range on the basis of a previously acquired integrated score for the question information, determine that the disease has worsened when the currently acquired integrated score for the question information is higher than the certain range, determine that the disease has improved when the currently acquired integrated score is lower than the certain range, and determine that the disease is maintained when the currently acquired integrated score is within the certain range. However, the present invention is not limited thereto.

FIG. 6 is a diagram illustrating a method of determining activity of a disease according to a second embodiment of the present invention.

Referring to FIG. 6, the computing device 100 may predict a change in activity of the user's disease on the basis of a key question for determining activity of the disease (S320). The computing device 100 may set at least one piece of question information corresponding to the user's disease as the key question. A question for best determining activity of the disease is preferably set as the key question but is not limited thereto.

The computing device 100 may compare an answer to the key question with a previously acquired answer to the key question and predict a change in the activity of the disease in accordance with the comparison result.

For example, when the key question is a question about a frequency of a symptom and answer information about the question is 1 to 3 times, 4 to 7 times, 7 to 12 times, and 20 times or more, the user may select the answer of 20 times or more. Meanwhile, when the user previously selected the answer of 7 to 12 times for the same question, the computing device 100 may predict that activity of the user's disease has increased. However, a type of key question and a type of answer list may vary, and types and a number of answers for predicting a change in activity of a disease in an answer list may also vary.

When it is predicted that the activity of the user's disease has not increased as a result of predicting a change in the activity of the user's disease (S321), the computing device 100 may determine that the user's disease has improved (S324).

Here, when the activity of the user's disease has improved, further analysis of answers may not be performed to increase efficiency in determining a result in terms of time and physical effort. Meanwhile, when there is no change in the activity, it may be determined that the user's disease has improved, but when there is no change in the activity a preset number of times or more, it may be determined that the user's disease has worsened. For example, when there is no change in the activity despite continuous treatment for the disease, the treatment may not be effective for the user. Accordingly, the user's disease may be determined to have worsened, and the user may be made to visit the doctor.

Meanwhile, when it is predicted that the activity of the user's disease has increased as a result of predicting a change in the activity of the user's disease (S321), the computing device 100 may finally determine a change in the activity of the user's disease on the basis of answers to auxiliary questions (S322).

When it is predicted on the basis of the key question that the activity of the disease has increased, the computing device 100 may confirm more accurately that the activity has increased by checking answers to the auxiliary questions. The auxiliary questions may include all question information other than the key question. In addition, the computing device 100 may classify the auxiliary questions into steps. In this case, whether the activity of the disease has increased may be determined on the basis of an answer to a first step auxiliary question. When it is determined that the activity of the disease has not increased, the user's disease may be determined to have improved, and when it is determined that the activity of the disease has increased, an answer to a second step auxiliary question may be checked. In this way, it is possible to reduce the load required for analyzing answers. In addition, classifying the auxiliary questions into steps may be classifying the auxiliary questions in descending order of suitability for determining a change in the activity of the disease.

When it is finally determined that the activity of the user's disease has not increased as a result of determining the activity of the user's disease using the auxiliary questions (S323), the computing device 100 may determine that the user's disease has improved (S324).

In addition, when it is finally determined that the activity of the user's disease has increased as a result of determining the activity of the user's disease using the auxiliary questions (S323), the computing device 100 may determine that the user's disease has worsened (S325).

According to another embodiment of the present invention, a measurement value may be acquired using a measurement device owned by the user in accordance with a type of the user's disease. For example, the measurement device may be, but is not limited to, a sphygmomanometer, a weight scale, a glucometer, or the like. The computing device 100 may judge a measurement value required for evaluating the activity of the user's disease in accordance with the type of disease and request the user to input the corresponding measurement value. The computing device 100 may provide an instruction to input a measurement value, and the user may input the measurement value acquired from the measurement device in accordance with the instruction provided via the user terminal 200.

The computing device 100 may compare the measurement value with a measurement value previously acquired from the user using the measurement device to determine activity of the user's disease.

The computing device 100 may periodically acquire a measurement value from the user. The computing device 100 may request a measurement value from the user at certain intervals, and every time a measurement value is acquired from the user, may evaluate activity of the disease and store the measurement value. Accordingly, the computing device 100 can compare a newly acquired measurement value with a previously measured measurement value. When the newly acquired measurement value is smaller or larger than a previously measured measurement value in accordance with a type of parameter measured by the measurement device, the computing device 100 may determine that the activity has changed. For example, when a blood pressure value is acquired from a sphygmomanometer, the computing device 100 may compare the current blood pressure value with a previously acquired blood pressure value. When the current blood pressure value is lower than the previous blood pressure value, it may be determined that the user's disease has improved, and when the current blood pressure value is higher than the previous blood pressure value, it may be determined that the user's disease has worsened. In the case of blood pressure, lower values indicate a safer condition. Accordingly, when the current blood pressure value is lower than the previous blood pressure value, it may be determined that the disease has improved, and when the current blood pressure value is higher than the previous blood pressure value, it may be determined that the disease has worsened. However, in the case where higher measurement values are safer when a current measurement value is lower than a previous measurement value, it may be determined that the disease has worsened, and when the current measurement value is higher than the previous measurement value, it may be determined that the disease has improved.

In addition, the computing device 100 may set a certain range on the basis of previously acquired measurement values, determine that the disease has worsened when the currently acquired measurement value is higher than the certain range, determine that the disease has improved when the currently acquired measurement value is lower than the certain range, and determine that the disease is maintained when the currently acquired measurement value is within the certain range. However, the present invention is not limited thereto. According to this embodiment, in the case where higher measurement values are safer as described above, when a current measurement value is higher than the certain range, it may be determined that the disease has improved, and when the current measurement value is lower than the certain range, it may be determined that the disease has improved.

In addition, the computing device 100 may merge a method of determining activity of a disease on the basis of answers to question information about the disease with a method of determining activity of a disease using a measurement value, thereby determining activity. For example, the computing device 100 may request the user to input a measurement value using the key question, calculate a score for the measurement value, and integrate the calculated score with a score calculated from answers to question information.

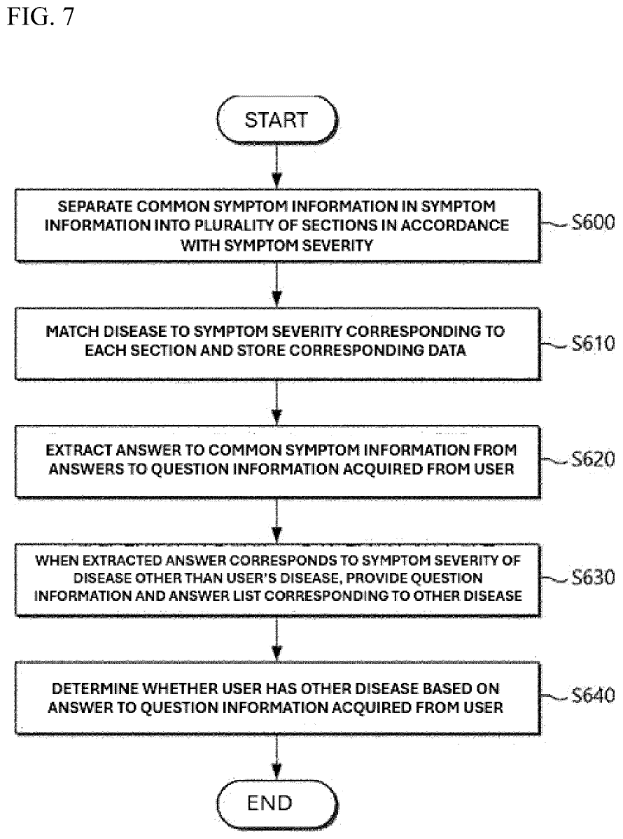

FIG. 7 is a diagram illustrating a method of determining whether a user has a disease according to another embodiment of the present invention.

Referring to FIG. 7, the computing device 100 may classify common symptom information in symptom information into a plurality of sections in accordance with symptom severity (S600). In other words, the computing device 100 may generate question information and answer information in accordance with a plurality of sections associated with a period and a frequency of a symptom. Meanwhile, common symptoms may be classified into a plurality of sections in accordance with symptom severity of each disease, but the present invention is not limited thereto. For example, both asthma and obstructive pulmonary disease have a symptom of shortness of breath. In the case of asthma, intermittent asthmatic attacks lead to shortness of breath, but in the case of obstructive pulmonary disease, shortness of breath continues. Accordingly, the computing device 100 may separate sections including a section corresponding to severity for determining asthma and a section corresponding to severity for determining obstructive pulmonary disease. However, the present invention is not limited thereto. A section without any symptom may be included, and even when a section corresponds to each disease, the section may include a plurality of sections in accordance with a condition. For example, a section corresponding to severity of asthma may be divided into a section corresponding to severity of less severe asthma and a section corresponding to severity of more severe asthma.

The computing device 100 may match a disease to symptom severity corresponding to each section and store the corresponding data (S610).

The computing device 100 may extract an answer to question information corresponding to common symptom information from answers to question information acquired from the user (S620). For example, when the user's disease is asthma, the computing device 100 may provide question information corresponding to asthma, collect question information corresponding to shortness of breath which is common symptom information to obstructive pulmonary disease, and extract the user's answer to the question information.

When the extracted answer corresponds to symptom severity of a disease other than the user's disease, the computing device 100 may provide question information and an answer list corresponding to the other disease to the user (S630).

For example, in the case of asthma, shortness of breath may occur once or twice a week, but in the case of obstructive pulmonary disease, shortness of breath may occur every day. Meanwhile, when the user with asthma is asked how many times he or she has experienced shortness of breath and responds that it happens every day, the computing device 100 may predict that the user has obstructive pulmonary disease. Accordingly, to determine whether the answer corresponds to a transitory symptom or obstructive pulmonary disease, the computing device 100 may provide question information and answer lists corresponding to obstructive pulmonary disease and acquire answers to the question information.

The computing device 100 may determine whether the user has the other disease on the basis of answers to the question information acquired from the user (S640). The computing device 100 may determine whether the user has the other disease using the same methods as in the above-described embodiments. However, the computing device 100 may store a reference value corresponding to a judgment criterion and determine whether the disease has developed on the basis of the reference value. For example, scores of the answers to question information may be integrated to determine that the disease has developed when the integrated score is the reference value or more, and determine that the disease has not developed when the integrated score is less than the reference value.

The computing device 100 according to an embodiment of the present invention may provide a result of determining the activity of the disease to the user. The computing device 100 may provide the activity of the disease to the user via the user terminal 200 such that the user can recognize whether the disease has improved or worsened. Further, the computing device 100 may provide description of the disease and description of symptoms of the disease or symptoms that may happen when the disease worsens.

In addition, the computing device 100 may provide the result of determining the activity of the disease to a hospital or a doctor designated by the user. When it is determined that the user's disease has worsened, the computing device 100 may provide a service for making an appointment with a hospital such that the user can see a doctor or receive treatment.

The computing device 100 may provide an associated doctor or hospital or a list of hospitals registered with a website or the like to the user. The user may select one hospital in the list, and the computing device 100 may provide a phone number or homepage address of the hospital to the user. Meanwhile, when the user has a physician in charge, the computing device 100 may provide the result of determining the activity of the disease to the selected physician in charge. This may be provided using the physician's phone number, identification (ID), email address, or the like acquired from the user's input, but the present invention is not limited thereto.

As described above, according to embodiments of the present invention, it is possible to provide a service provision method, device, and program for determining activity of a disease in which activity of a user's disease is evaluated on the basis of answers to question information for evaluating activity of the disease and a doctor can rapidly intervene in accordance with the activity.

Embodiments of the present invention have been described above with reference to the accompanying drawings, but those skilled in the technical field to which the present invention pertains should understand that the present invention can be implemented in other specific forms without changing the technical spirit or essential features thereof. Therefore, it should be understood that the embodiments described above are illustrative in all aspects and are not restrictive.

### DESCRIPTION OF SIGNS

100: computing device
200: user terminal
300: external server

1. A service provision method for determining activity of a disease performed by a computing device, the method comprising:
acquiring information on a user's disease;
collecting symptom information about various diseases;
generating a plurality of pieces of question information and a plurality of answer lists for evaluating activity of each of the various diseases on the basis of the collected symptom information;
providing the user with question information and answer lists corresponding to the user's disease; and
determining activity of the user's disease on the basis of answers to the question information acquired from the user.

2. The service provision method of claim 1, wherein the generating of the plurality of pieces of question information and the answer lists comprises:
extracting symptom information common to several diseases from symptom information about the various diseases;
generating question information about symptom information not common to several diseases and the common symptom information in the symptom information;
generating a plurality of answer lists corresponding to the question information; and
classifying the generated question information and the answer lists by disease.

3. The service provision method of claim 2, wherein the determining of the activity of the user's disease comprises:
receiving at least one selected from a plurality of answers to each piece of the question information from the user;
when answers to the question information are input, calculating a score corresponding to an answer selected for each piece of the question information and integrating the calculated scores; and
comparing the integrated score with a score integrated on the basis of answers to the question information previously acquired from the user or a reference score for each disease and determining activity of the user's disease in accordance with a comparison result.

4. The service provision method of claim 3, wherein the integrating of the calculated scores comprises:
extracting weights set for the question information;
calculating scores by applying the weights set for the question information in accordance with diseases to scores corresponding to the selected answers to the question information; and
integrating the scores to which the weights are applied.

5. The service provision method of claim 1, wherein the determining of the activity of the user's disease comprises:
predicting a change in the activity of the user's disease on the basis of an answer to a key question for determining the activity of the disease; and
when the activity of the user's disease is predicted to increase, determining a change in the activity of the user's disease,
wherein the final determining of the change in the activity of the disease further comprises determining that the user's disease has worsened when the activity of the disease is determined to have increased as a result of analyzing answers to auxiliary questions, and determining that the user's disease has improved when the activity of the disease is determined to have decreased.

6. The service provision method of claim 2, wherein the generating of the plurality of pieces of question information and the plurality of answer lists comprises:
separating the common symptom information into a plurality of sections in accordance with symptom severity; and
matching a disease to symptom severity corresponding to each section and storing corresponding data, and
the determining of the activity of the user's disease comprises:
extracting an answer to the question information corresponding to the common symptom information from the answers to the question information acquired from the user;
when the extracted answer corresponds to symptom severity of a disease other than the user's disease, providing question information and an answer list corresponding to the other disease to the user; and
determining whether the user has the other disease on the basis of an answer to the question information acquired from the user.

7. The service provision method of claim 1, wherein the acquiring of the information on the user's disease further comprises:
acquiring a measurement value using a measurement device owned by the user in accordance with a type of the user's disease, and
the determining of the activity of the user's disease further comprises comparing the measurement value with a measurement value previously acquired from the user using the measurement device to determine activity of the user's disease.

8. The service provision method of claim 1, further comprising:
providing the determined activity of the disease to the user; and
providing the determined activity of the disease to a hospital or a doctor designated by the user in accordance with the activity of the disease.

9. A device, comprising:
a memory configured to store one or more instructions; and
a processor configured to execute the one or more instructions stored in the memory,
wherein the processor performs the method of claim 1 by executing the one or more instructions.

10. A computer program stored in a computer-readable recording medium to perform the method of claim 1 in combination with a computer which is hardware.
